(19) United States
(12) Patent Application Publication
(10) Pub. No.: US 2006/0188543 A1
(43) Pub. Date: Aug. 24, 2006

(54) Nanoparticle coating for drug delivery

(76) Inventor: Si-Shen Feng, Singapore (SG)

Correspondence Address: DICKSTEIN SHAPIRO MORIN & OSHINSKY LLP, 2101 L Street, NW, Washington, DC, 20037, US

(21) Appl. No.: 11/342,662

(22) Filed: Jan. 31, 2006

Related U.S. Application Data
(60) Provisional application No. 60/648,978, filed on Jan. 31, 2005.

(51) Int. Cl. A61F 2/00

(52) U.S. Cl. 424/423; 977/906

(57) ABSTRACT

Coatings for drug delivery. In particular, coatings comprising nanoparticles loaded with at least one drug, on implants such as stents, to deliver drugs at the sites of implantation.

### CROSS-REFERENCE TO RELATED APPLICATION

This application claims the benefit of U.S. Provisional Application No. 60/648,978, filed on Jan. 31, 2005, the entirety of the contents of which are hereby incorporated by reference herein.

### FIELD OF THE INVENTION

This invention relates to coatings for drug delivery. In particular, the invention relates to coatings comprising nanoparticles loaded with at least one drug.

### BACKGROUND OF THE INVENTION

Coronary atherosclerosis and heart attacks are the leading cause of human mortality. The 2001 deaths were 28.2% in Singapore and 28.9% in the USA. Although great progress has been made in the past 50 years with the death rate deceased from 586.8/100,000 in 1950 to 245.8/100,000 in 2001, cardiovascular diseases are still the number one killer all over the world. The most common treatments for cardiovascular diseases include so far the percutaneous transluminal coronary angioplasty (PTCA) with or without an intracoronary stent. Although efficient, this kind of treatments is far from satisfactory. About 30-50% of the patients treated with PTCA would experience restenosis within 3-6 months (Popma et al, 1991).

Restenosis is the re-obstruction of the coronary arteries, which is triggered by blood vessel wall injury caused by intervention to relieve arterial obstruction. Restenosis is a complex process caused by many factors such as elastic recoil of vessels after dilation, proliferation and migration of vascular smooth muscle cells (VSMCs), enhanced extracellular matrix (ECM) synthesis, blood vessel wall remodeling, thrombus formation (Huudenschild C C, 1993). There are two kinds of treatment for restenosis so far: mechanical treatment and drug therapies. The former is stenting. Although popular, stenting does not solve the problem. Some 40% of the patents treated with stent will suffer from restenosis again within six months. The later includes the treatment by antiproliferative, antiplatelet, anticoagulant agents such as paclitaxel, calcium channel antagonists, inhibitors of angiotensin converting enzyme, corticosteroids, fish oil diet. However, how to delivery the drugs to VSMCs is still a problem. One method is to use paclitaxel-eluting stents to prevent restenosis following implantation of the stent (Liistro et al, 2002).

Paclitaxel is one of the best antineoplastic drugs found from nature in the past decades. It has excellent therapeutic effects against a wide spectrum of cancers (Wani et al, 1971). It was approved by FDA for ovarian cancer in 1992, for advanced breast cancer in 1994 and for early stage breast cancer in October 1999.

The mechanism of action of paclitaxel has been intensively investigated. It inhibits mitosis in tumor cells by binding to microtubules. Paclitaxel aids polymerization of tubulin dimmers to form microtubules and thus stabilizes the microtubules leading to cell death (Lopes et al, 1993). Although effective, paclitaxel and other antiproliferative drugs have formulation problems in their clinical applications. The only dosage form of paclitaxel so far is Taxol®, which was developed by Bristol-Myers Squibb (BMS) Company. Taxol® uses Cremophor EL as adjuvant, which has been found to be responsible for many severe side effects including hypersensitivity reactions, nephrotoxicity, neurotoxicity and cardiotoxicity, some of them being life-threatening (Dorr, 1994).

As such, while paclitaxel-eluting stents are being used to prevent restenosis following implantation of the stent, problems still persist mainly in the form of the side effects due to the adjuvant used.

Accordingly, there is a need in the art for the development of new and/or improved mechanical treatments and/or drug therapies for antineoplastic and/or antiproliferative treatments which overcome or at least ameliorate the limitations and/or problems of the prior art.

### SUMMARY OF THE INVENTION

Accordingly, in one aspect, the present invention provides a coating comprising at least one type of nanoparticle, wherein the at least one type of nanoparticle is emulsified by at least one amphiphilic emulsifier and loaded with the at least one drug. The at least one type of nanoparticle may be made of biodegradable and/or bioresorbable polymer. For example, the nanoparticle may be made of PLGA. Preferably, the emulsifier is vitamin E TPGS. The drug may be paclitaxel. The coating may be applied on a surface. In particular, the coating may be applied on an implant. The implant may be a stent. For example, a cardiovascular stent.

In another aspect, the present invention provides an implant coated with at least one coating, the coating comprising at least one type of nanoparticle, wherein the at least one type of nanoparticle is emulsified by vitamin E TPGS and loaded with at least one drug. The implant may be a stent. For example, a cardiovascular stent. The implant may be an implant for brachytherapy. The drug may be radioactive material and/or a chemotherapy drug.

In another aspect, the present invention provides a process of coating an implant, the process comprising:
(a) providing an implant;
(b) providing a lipid monolayer comprising at least one type of nanoparticle, the nanoparticle being emulsified with vitamin E TPGS and loaded with at least one drug; and
(c) coating the implant with the lipid monolayer.

In another aspect, the present invention provides a method of controlling and/or reducing restenosis and/or multi-drug resistence in a subject receiving a cardiovascular stent, the method comprising implanting a cardiovascular stent coated with least one coating comprising at least one type of nanoparticle made of a biodegradeable and/or bioresorbable polymer, the at least one type of nanoparticle being emulsified by vitamin E TPGS and loaded with paclitaxel.

In another aspect, the present invention provides an implant for brachytherapy, the implant comprising: at least one type of nanoparticles, wherein the at least one type of nanoparticle is emulsified by vitamin E TPGS and loaded with the at least one drug.

### ABBREVIATIONS USED

PLGA: poly (lactic-co-glycolic acid).
Vitamin E TPGS, or TPGS: d-α-tocopheryl polyethylene glycol 1000 succinate.
PVA: polyvinyl alcohol.
DSC: differential scanning calorimetry.
SEM: scanning electron microscopy.
AFM: atomic force microcopy.
FTIR-PAS: Fourier transform infra-red photoacoustic spectroscopy.
XPS: X-ray photoelectron spectroscopy.
FDA: The US Food and Drug Administration.
PBS: Phosphate buffered saline.
DCM: dichloromethane.
VSMC: vascular smooth muscle cell

### BRIEF DESCRIPTION OF THE DRAWINGS

FIG. 1. Chemical structures of Vitamin E TPGS and Vitamin E.

FIG. 2. Particle size distribution of nanoparticles (1. Vitamin E TPGS added in water; 2. Vitamin E TPGS+PVA as emulsifier; 3. Vitamin E TPGS added in oil; 4. PVA as emulsifier).

FIG. 3 (A,B,C,D). SEM images of Paclitaxel loaded PLGA nanoparticles (A: Vitamin E TPGS added in water; B: Vitamin E TPGS+PVA as emulsifier; C: Vitamin E TPGS added in oil; D: PVA as emulsifier).

FIG. 4 (A,B,C,D). AFM images of nanoparticles prepared by using TPGS as emulsifier.

FIG. 5. DSC thermograms of 1) 100% Paclitaxel; 2) Physical mixture of 10% Paclitaxel and 90% PLGA; 3) Paclitaxel loaded nanoparticles with TPGS added in water; 4) Paclitaxel loaded nanoparticles TPGS+PVA as emulsifier; 5) Paclitaxel loaded nanoparticles with TPGS added in oil.

FIG. 6 (A,B,C). XPS analysis of paclitaxel-loaded PLGA nanoparticles, which are emulsified by TPGS or PVA.

FIG. 7 (A,B). FTIR-PAS analysis of paclitaxel-loaded PLGA nanoparticles, which are emulsified by TPGS or PVA.

FIG. 8. In vitro drug release of paclitaxel-loaded PLGA nanoparticles, which are emulsified by TPGS or PVA.

FIG. 9. Confocal microscopic image of cardiovascular smooth muscle cells after exposed to vitamin E TPGS emulsified, Coumarin-6 loaded nanoparticles for 1 hr at 37° C., followed by nucleus staining using propidium iodide.

FIG. 10. Cryo-SEM image of a cross-section of a typical vascular smooth muscle cell after 1 hour incubation at 37° C. with vitamin E TPGS-emulsified, paclitaxel-loaded PLGA nanoparticles The arrows indicate some nanoparticles found throughout the endoplasm and around the nucleus. Some nanoparticles were found adsorbed on the cell membrane.

FIG. 11. Plasma concentration-time profiles of paclitaxel formulated in Taxol® (paclitaxel) (10 mg/kg) or TPGS-emulsified PLGA nanoparticles (10 mg/kg as well as 40 mg/kg) after intravenous administration to male Sprague-Dawley rats (180-200 gm and 4-5 week old). The paclitaxel-loaded nanoparticles and paclitaxel (Taxol®) doses were dispersed or diluted with saline and administrated through the tail vein at the same paclitaxel dose of 10 mg/kg body weight. Blood samples were collected at intervals and the plasma extracted for HPLC or LC/MS/MS analyses. The concentrations between the side-effect level (8,540 ng/ml) and the minimum-effective level (43 ng/ml) show the therapeutic window of the drug.

### DEFINITIONS

Coating—at least one layer of a chemical or pharmaceutical composition applied to at least one surface of an insoluble (for example, a solid) object, for example, a support, or product. The support may be an implant, for example, a stent.

Drug—Ac active principle, a compound, a medicament and/or a pharmaceutical composition suitable to be administered to a subject to obtain a desirable medical outcome.

Implant—An object implantable and/or emplaced into a subject.

Load/Loading—to add a drug to a carrier, for example, to add paclitaxel to a nanoparticle.

Nanoparticle—A particle whose size is in the nanometer range of 50 nm to 1,000 nm, preferably from 100 nm to 800 nm, from 150 nm to 500 nm and from 200 nm to 400 nm. When loaded with the drug, the nanoparticles may be referred to as nanospheres and the nanospheres may be in the preferred range of 300 nm to 600 nm.

Biodegradable—The quality of being able to break down in the body of a subject.

Bioresorbable—The quality of being resorbed in or by the body. The terms biosorbable, bioresorbable and bioabsorbable are used interchangeably in the present application.

### DETAILED DESCRIPTION OF THE INVENTION

Bibliographic references mentioned in the present specification are for convenience listed in the form of a list of references and added at the end of the examples. The whole content of such bibliographic references is herein incorporated by reference.

The present invention relates to coatings for drug delivery. In particular, the invention relates to a least one coating comprising nanoparticles loaded with at least one drug. More in particular, the inventions relate to a coating comprising at least one type of nanoparticle, wherein the at least one type of nanoparticle is emulsified by an amphiphilic emulsifier and loaded with at least one drug. Preferably, the emulsifier is vitamin E TPGS. There is also provided an implant coated with the coating according to the invention. The implant may be an implant such as a stent, to deliver drugs at the sites of implantation. The stent may be a cardiovascular stent. The nanoparticle may be made of a biodegradable and/or bioresorbable polymer, for example PLGA.

This invention also provides a novel kind of stent suitable for treatment of cardiovascular restenosis, which may represent the fourth generation of stents versus the nude stents as the first generation, the drug-eluting stents as the second generation, and the polymeric matrix coated stents for controlled drug release as the third generation. It is not a simple or obvious continuation of the first three generations of the cardiovascular stents. The nanoparticle formulation of the antiproliferative, antiplatelet, anticoagulant agents and the nanoparticle coating techniques represent the features which may distinguish the stent according to the invention from any other kinds of stents, In the stents of the prior art, the cellular uptake of the drugs, either formulated in adjuvant or released from polymeric matrix, are not efficient.

The drugs in this invention, instead, are carried by nanoparticles, which serve as a drug reservoir and they may be used as a controlled drug delivery, even when taken up into the cells. The nanoparticle-coated stents can thus result in much lower incidence of restenosis than stents of the previous three generations.

The present invention also relates to radioactive pellets for brachytherapy or short-term internal radiotherapy, for treatment of tumors. The invention may be a coating comprising at least one radioactive material and at least one type of biogradable polymer nanoparticle. Such coatings may be used to coat small implants or pellets for insertion into a cancer tumor to kill tumor cells.

Cardiovascular Stents Coated by Nanoparticles. In a specific embodiment, the present invention provides cardiovascular stents coated with drug-loaded, vitamin E TPGS-emulsified nanoparticles of biodegradable polymers. Such a coated stent can result in high cellular uptake of the drug and thus low viability of vascular smooth muscle cells (VSMC), thus attaining better effects in preventing restenosis compared to cardiovascular stents of the prior art. The nanoparticles serve the function of a reservoir for sustained and controlled release of the encapsulated drug after being taken up by VSMCs. Standard cardiovascular stents are commercially available at much lower price than that of the drug-eluting stents from the Boston Scientific Inc. They can be used for further process by this invention. The raw stents can then be coated by a suitable technique such as the modified dipping technique developed in this invention. This technique is similar to the Langmuir-Blodgett deposition technique, which is often used to obtain of a piece of lipid monolayer deposited the air-water interface into a solid surface such as a flat piece of mica. The dipping starts either from the air phase for stents which have hydrophobic surface (e.g. polymeric), or from the water phase for those which have hydrophilic surface (e.g. metal). The dipping can be repeatedly carried out until a desired amount of drug has been contained in the multi-layers of the lipid-nanoparticle mixture. However, the dipping process should be finished with the lipid head group surface if the coated stents are to be restored in a liquid phase, or with the lipid chain layer if the coated stents are to be restored in a dry condition. It can be shown that the coated layers can be quite stable under room temperature.

Method for Preventing Restenosis. A person skilled in the art will appreciate that the cardiovascular stent as taught in above may be used in a method to minimize restenosis or multi-drug resistence in a subject receiving a cardiovascular stent. The method may comprise implanting a cardiovascular stent comprising least one coating comprising at least one type of nanoparticle made of a biodegradeable polymer, the at least one type of nanoparticle loaded with paclitaxel where the at least one type of nanoparticle was emulsified by vitamin E TPGS.

Implant for Brachytherapy. In another specific embodiment, the present invention may be used as a temporary therapeutic implant for the treatment of a cancer such as breast cancer. A pellet of a suitable biodegradable polymer such as PLGA coated with nanoparticles of the same or different biodegradable polymer which have been emulsified by vitamin E TGPS and loaded with a suitable drug. Suitable drugs for treating tumors include radiotherapy or chemotherapy drugs. A suitable radioactive material is iridium and it can be incorporated into the nanoparticles under appropriate radiation protection conditions using the method of the present invention. Unlike brachytherapy implants (pellets or seeds) of the prior art, the brachytherapy implants under the present invention need not be removed after the course of treatment. The biodegradable and/or bioresorbable polymers and radioactive material may be formulated to degrade or decay (respectively) when the duration of therapy is over. It is envisaged that using this embodiment of the present invention, a much lower dose of radioactive material, perhaps one to several orders of magnitude lower, will be needed, compared to brachytherapy pellets of the current art. As an alternative to using a radioactive material, the pellet or implant may be instead loaded with a suitable anti-tumor or chemotherapy drug such as paclitaxel.

Variations under the Present Invention. A person skilled in the art will appreciate that many other variations of the present invention may be possible. Such variations may include, but are not be confined to:
local drug delivery by nanoparticles from a reservoir within the implant wall, such reservoirs may be formed by the implant wall having a porous structure;
local drug delivery by nanoparticles with balloon coated with drug-loaded nanoparticles during percutaneous transluminal coronary angioplasty; and
pulmonary delivery of drug-loaded nanoparticles to prevent cardiovascular restenosis;

It will be appreciated that various modifications and improvements can be made by a person skilled in the art without departing from the scope of the present invention.

Having now generally described the invention, the same will be more readily understood through reference to the following examples which are provided by way of illustration, and are not intended to be limiting of the present invention.

### EXAMPLES

Materials

Poly (DL-lactide-co-glycolide) (PLGA, 50:50, MW 40,000-75,000), polyvinyl alcohol (PVA, MW 30,000-70,000, the viscosity of a 4% solution was 4 to 6 cp (centipoise) at 20° C., with the degree of hydrolysis between 87 to 90 percent) were purchased from Sigma. Phospholipids such as DPPC (1,2-dipalmitoyl-sn-glycerol-3-phosphocholine) were purchased from Avanti Polar Lipid, Inc. (Alabaster, Ala., USA). Paclitaxel of 99.8% purity was purchased from Yunnan Hande Biotechnology Inc., China. Vitamin E TPGS was provided by Eastman Chemical Company, USA. The solvent methylene chloride (dichloromethane, DCM, analytical grade) was purchased from Mallinckrodt. Acetonitrile, used as mobile phase in HPLC, was purchased from EM Science (chrom AR HPLC grade). Distilled water produced by Millipore (Milli Q plus 185, Bedford, MX 01730, USA) was used throughout the experiment. The in vitro release measurement was carried out at pH 7.4 and 37° C. in phosphate buffered saline (PBS), which was purchased from Sigma Chemical Co. All other chemicals used were of reagent grade.

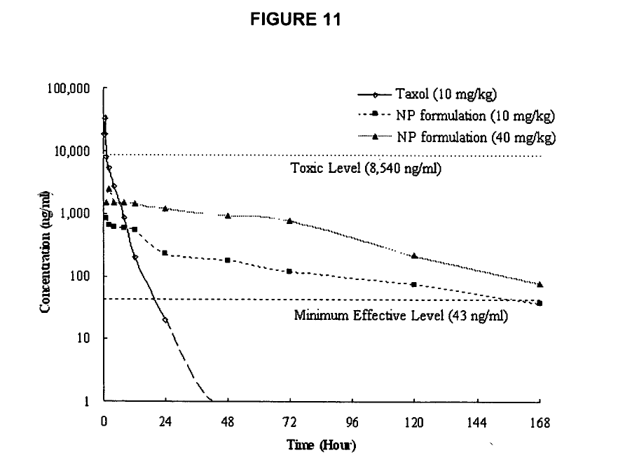

One feature of this invention is to use an amphiphilic emulsifier such as d-a-tocopheryl polyethylene glycol 1000 succinate (vitamin E TPGS, or TPGS) as the emulsifier in the drug-loaded nanoparticle preparation, which the inventor has found to have surprisingly much higher emulsification effects, drug encapsulation efficiency, higher cellular uptake, more advantageous pharmacokinetics (FIG. 11) and biocompatibility compared to other emulsifiers such as polyvinyl alcohol (PVA), which is used most often in particle technology (Win and Feng, 2005).

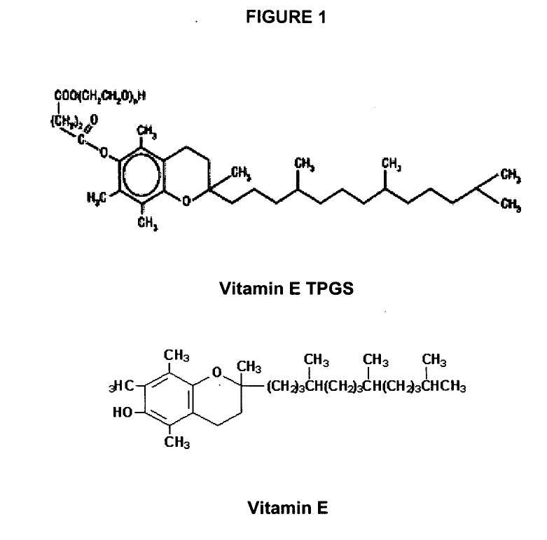

Vitamin E TPGS is a water soluble derivative of natural vitamin E, which is formed by esterification of vitamin E succinate with polyethylene glycol 1000. TPGS is a safe and effective form of vitamin E for reversing or preventing vitamin E deficiency. Vitamin E TPGS could be absorbed intact readily in the gastrointestinal tracts, and could inhibit P-glycoprotein in the intestine to enhance the cytotoxicity of anticancer drugs such as doxorubicin, vinblastine (a commonly used medication for HIV and AIDS patients), and paclitaxel. Vitamin E TPGS may also increase the absorption flux of amprenavir by enhancing its solubility and permeability, which are essential in the development of the soft gelatin capsule formulation for use in the clinic. The chemical structure of vitamin E TPGS (FIG. 1) comprises both lipophilicity and hydrophilicity, resulting in amphiphilic properties. Moreover, its lipophilic alkyl tail (polyethylene glycol) and hydrophilic polar head portion (tocopherol succinate) are bulky and have large surface areas. Such characteristic makes it a good emulsifier, which can emulsify a wide range of water-oil immiscible systems, thus enabling a wide range of drugs to be used under the present invention. The hydrophile/lipophile balance (HLB) of TPGS is about 13. It melts at 37-41° C. and is heat stable under temperature 200° C.

While vitamin E TPGS is cited as an example of a suitable emulsifier under the present invention, it is contemplated that other amphiphilic emulsifiers, particular natural emulsifiers, such as phospholipids and cholesterol, and their derivatives, may be used under the present invention.

To illustrate the invention, the biodegradable polymer employed to form nanoparticles in an embodiment of the present invention is the most widely used, FDA approved poly (D,L-lactic-co-glycolic acid) (PLGA) as its biodegradation, synthesis technique and application for drug delivery is well-known. Other suitable biodegradable and/or bioresorbable polymers may be used as well. Further, paclitaxel is used as an example of a drug to illustrate how the present example may be practiced. However, other drugs suitable for the purpose of the present invention may also be used.

Methods

Nanoparticle Preparation

The nanoparticles with or without paclitaxel were prepared by the solvent evaporation/extraction technique (the single emulsion technique). Typically, 200 mg of PLGA was dissolved in DCM. The solution of organic phase was slowly poured into the stirred aqueous solution of PVA or TPGS and sonicated with energy output of 50 w in a pulse mode (Misonix Incorporated).

To incorporate paclitaxel, paclitaxel at about 10-20% of the nanoparticle weight, and PLGA are added in DCM in the same time with the nanoparticles. Alternatively, paclitaxel and PLGA can be separately dissolved in a small amount of DCM and then subsequently combined.

The oil-in-water (O/W) emulsion thus obtained was gently stirred at room temperature (22° C.) by a magnetic stirrer (EYELA Magnetic stirrer RC-2) for overnight to evaporate the organic solvent. The resultant sample was collected by centrifugation (Eppendorf of model 5810R, 8000-9000 rpm, 10 min, 16° C.) and washed with distilled water at least 4 times to remove the emulsifier. The produced suspension was freeze-dried (Christ, Alpha-2, Martin Christ) to obtain a fine powder of nanoparticles, which was placed and kept in vacuum desiccators. The loading ratio of paclitaxel for the preparation was around 10%.

Characterization of Nanoparticles

(a) Size and Size Distribution.

The particle size and size distribution of the prepared nanoparticles were measured by the laser light scattering (Brookhaven Instruments Corporation 90 Plus Particle Sizer). The dried powder samples were suspended in deionised water and sonicated briefly before measurement. The obtained homogeneous suspension was determined for the volume mean diameter, size distribution and polydispersity.

TABLE 1
Size, size distribution and drug encapsulation efficiency (EE)
of paclitaxel loaded PLGA nanoparticles (drug loading = 10%).

| | Mean | | |
|---|---|---|---|
| | Diameter | Poly- | EE |
| Samples | (nm) ± SD | dispersity | (%) |
| 1. Nanospheres with TPGS added in | 685 ± 39 | 0.005 | 100 |
| water | | | |
| 2. Nanospheres with TPGS + | 485 ± 83 | 0.005 | 53 |
| PVA as emulsifier | | | |
| 3. Nanospheres with TPGS added in oil | 796 ± 136 | 0.045 | 100 |
| 4. Nanospheres with PVA as emulsifier | 695 ± 39 | 0.005 | 58 |

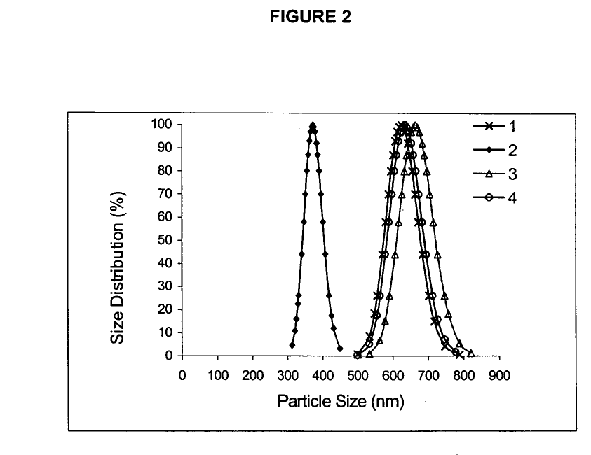

Table 1 shows the size, size distribution and drug encapsulation efficiency (EE) of four batches of samples of TPGS or PVA emulsified, paclitaxel loaded PLGA nanoparticles (or nanospheres). The dug loading is 10%. Their size is as desired in the range of 300-600 nm for stent coating. They are quite uniform withy small polydispersity of 0.005 to 0.045. 100% drug encapsulation efficiency has been achieved for the first time in the literature. The paclitaxel encapsulation efficiency in nanoparticle formulation in others' work is usually 40-60%. FIG. 2 shows the nanoparticle size distribution, which is obtained from laser light scattering measurement.

(b) Drug Encapsulation Efficiency Measurement.

The drug entrapped in the nanoparticles was determined in triplicate by HPLC (Agilent LC1100). A reverse phase Inertsil â ODS—3 column (150 4.6 mm i.d., pore size 5 μm, GL Science Inc, Tokyo, Japan) was used. To obtain the solution for analysis, 3 mg of nanoparticles was dissolved in 1 ml of DCM and 5 ml of acetonitrile-water (50:50) was then added. A nitrogen stream was introduced to evaporate the DCM till a clear solution was obtained. The solution was put into vial for HPLC to detect the paclitaxel concentration. The mobile phase consisted of a mixture of acetonitrile and water (50:50, v/v), and was delivered at a flow rate of 1.00 ml/min with a pump (HP 1100 High pressure Gradient Pump). A 100 μl aliquot of the sample was injected with an autoinjector (HP 1100 Autosampler). The column effluent was detected at 227 nm with a variable wavelength detector (HP 1100 VWD). The calibration curve for the quantification of paclitaxel was linear over the range of standard concentration of paclitaxel at 60-60,000 ng/ml with a correlation coefficient of R2=1.0. The solvent for calibration is the mixture of acetonitrile and water (50:50, v/v).

As inefficient extraction may exist, a correction of the calculated encapsulation efficiency is needed. The recovery efficiency factor of the extraction procedure on encapsulation efficiency was determined according to the following method. A certain weight of pure paclitaxel which was similar to the amount loaded in a certain amount of nanoparticles and 3.0-5.0 mg of placebo nanoparticles or polymer were dissolved in 1 ml of DCM. 5 ml of acetonitrile-water (50:50) was added. The same extraction procedure as described above was done. The resulted factor was 100%, which means that about 100% of the original amount of the paclitaxel could be detected. The encapsulation efficiency of paclitaxel was obtained as the mass ratio between entrapped amount of paclitaxel in nanoparticles and the drugs used in the preparation.

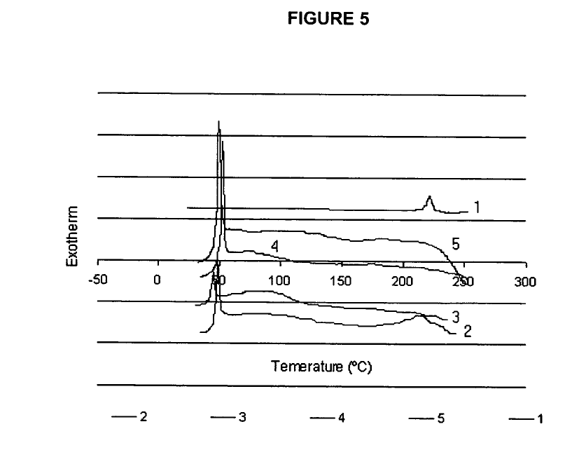

The encapsulation efficiency of the four recipes was illustrated in FIG. 5 and listed in Table 1, from which the most notable success by employing vitamin E TPGS as emulsifier could be concluded. The percentage of entrapped paclitaxel in the nanoparticles could reach as high as 100 (sample 1 and sample 3) as emulsified by TPGS.

This achievement significantly improves the solvent evaporation/extraction technique for fabrication of nanoparticles. It is normally difficult to approach such a highly entrapped efficiency. The droplet formation, droplet stabilisation, nanoparticles hardening is the three essential stages of nanoparticles formation. The formation of solid nanoparticles is brought about by the diffusion of the solvent from the emulsion droplet into the continuous phase, followed by the evaporation/extraction of the volatile solvent and the simultaneous inward diffusion of the nonsolvent into the droplet. During this course, a partition occurs across the interface from the dispersed phase to the continuous phase. However, the partition is not limited to the organic solvent, both the polymer and the drug molecules may also partition or diffuse across this interface from the organic phase toward the external aqueous phase. The partitioning phenomenon between the dispersed and the dispersing phases contributes to a substantial lowering of microencapsulation yield as well as the encapsulation efficiency.

Although the physicochemical characteristic of the drug molecule plays an important role, the surfactant character also has significant effect on the localisation of the drug molecule. Modifying the dispersed or dispersing phase of the emulsion by the emulsifier/stabiliser to reduce the leakage of the drug molecule from the oily droplets can thus make improvement of the encapsulation efficiency of the drug in the nanoparticles. In the present case, the bulky and large surface area of TPGS resulting from its big lipophilic alkyl tail (polyethylene glycol) and hydrophilic polar head portion (tocopherol succinate) could effectively protect the diffusion or partition of the hydrophobic paclitaxel from polymer to external phase. The encapsulation efficiency of paclitaxel in the polymeric nanoparticles can thus be significantly improved. Besides, as a novel surfactant stabiliser, which can be added either in the aqueous phase or in the oil phase, the TPGS can always result in a very high encapsulation efficiency, which cannot be achieved by PVA. When PVA was added together with the TPGS (sample 2), the entrapped efficiency was lowered to a level, which is the same low for the PVA emulsified nanoparticles (sample 4). This result shows the shortage of PVA as emulsifier.

(c) Morphology

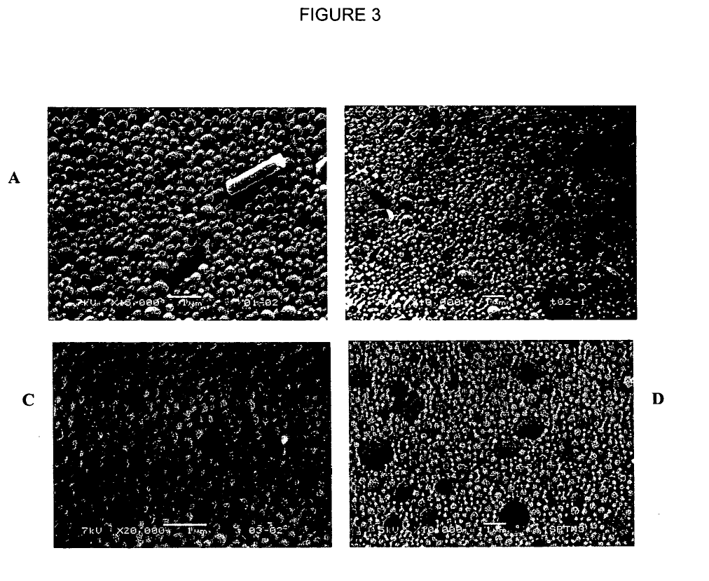

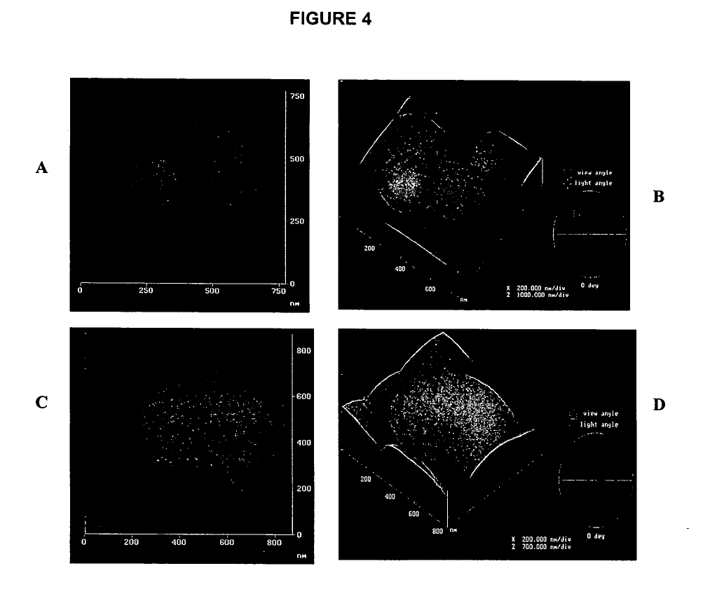

FIGS. 3 and 4 showed the SEM and AFM images of the nanoparticles. There were no significant differences in morphology, which can be observed, among the four recipes fabricated with vitamin E TPGS and PVA as emulsifier respectively. All nanoparticles were in fine spherical shape with smooth surfaces and without any aggregation or adhesion. In fabrication of paclitaxel loaded nanoparticles by applying the solvent evaporation/extraction technique, the use of surfactant stabilizer is necessary to stabilize the dispersed-phase droplets and inhibit coalescence. The amphipathic surfactants align themselves at the droplet surface, so promoting stability by lowering the free energy at the interface between the two phases and resisting coalescence and flocculation of the nanoparticles. Surfactants employed in the o/w process tend to be hydrophilic in nature, and among them by far, PVA is the most widely used and would appear to be the most effective for formation of micro or nanoparticles (Huudenschild, 1993).

The inventor made a surprising discovery that vitamin E TPGS is an ideal emulsifier compared to emulsifiers of the prior art such as PVA for the preparation of polymeric nanoparticles by the solvent evaporation/extraction technique. As a surfactant stabiliser, vitamin E TPGS possesses all merits of PVA as emulsifier. However, it is a better, more effective emulsifier. One advantage of vitamin E TPGS against PVA is its unique property of being able to dissolve both in water and in oil. No matter it was added in the water phase (sample 1) or in the oil phase (sample 3), similar properties of nanoparticles could be obtained, as indicated in Table 1 and FIGS. 2 to 4. The smaller size of sample 2 (Table 1 and FIG. 2) prepared by using the PVA and TPGS together as emulsifier may result from the additive effect of the co-surfactant. In addition, the suspending stability of the vitamin E TPGS emulsified nanoparticles was similar to that of PVA emulsified nanoparticles. To collect the samples of both types, a centrifugation of at least 8000 rpm was needed so that the nanoparticles could be precipitated at the bottom of the tubes. Similarly, nanoparticles of both types could be suspended stably in PBS buffer solution as well as in the buffer containing bovine serum albumin (BSA). To collect the nanoparticles, centrifugation of more than 8000 rpm was also needed.

(d) DSC Analysis

The physical status of the paclitaxel inside the nanoparticles was characterized by the differential scanning calorimetry (DSC) thermogram analysis (DSC, 2920 Modulated, Universal V2.6D TA instruments). 8 mg of sample was sealed in standard aluminum pans with lids. The samples were purged with pure dry nitrogen at a flow rate of 40 ml/min. A temperature ramp speed was set at 10° C./min and the heat flow was recorded from 0 to 250° C. Indium was used as the standard reference material to calibrate the temperature and energy scales of the DSC instrument. DSC analysis of pure paclitaxel was previously carried out to identify the melting point peak. As a control the physical mixtures of paclitaxel and placebo nanoparticles of 1% and 10% paclitaxel proportion were analysed to observe the change of the melting endotherm of crystallized paclitaxel in the mixture. Subsequently, the nanoparticles with 10% loading level of paclitaxel were analysed as needed by the sensitivity of the apparatus.

FIG. 5 showed the DSC thermogram analysis, which provided qualitative and quantitative information about the physical status of the drug in the nanoparticles and the control samples, which are the pure drug and the physical mixture of pure paclitaxel and placebo nanoparticles. The pure paclitaxel showed an endothermic peak of melting at 223.0° C. (sample 1), which was broadened and shifted to a lower temperature at about 218.0° C. (sample 2) for the 10% paclitaxel-placebo nanoparticles physical mixture. There was no peak observed at the temperature range of 150° C.-250° C. for the placebo nanoparticles and all drug loaded nanoparticles (sample 3, 4, 5). The DSC experiment didn't detect any crystalline drug material in the nanoparticles samples. It can thus be concluded that the paclitaxel formulated in the four batches of nanoparticles was in an amorphous or disordered-crystalline phase of a molecular dispersion or a solid solution state in the polymer matrix after the fabrication. Moreover, the glass transition temperature of the polymer PLGA employed in all the four batches of nanoparticles wasn't influenced obviously by the procedure, which meant that the surfactant stabilizer did not influence the thermogram property of polymeric material significantly.

TABLE 2
XPS (C1s) analysis of paclitaxel-loaded, PLGA nanoparticles
which are emulsified by TPGS or PVA respectively.

| | XPS elemental | | | XPS C1s envelope | | |
|---|---|---|---|---|---|---|
| | ratio (%) | | | ratios (%) | | |
| Samples | C | O | N | C—C/C—H | C—O | O—C═O |
| PLGA | 34.6 | 65.0 | 0.0 | 52.0 | 30.0 | 18.0 |
| PVA | 68.0 | 32.0 | 0.0 | 49.9 | 42.7 | 7.4 |
| TPGS | 69.1 | 30.9 | 0.0 | 57.7 | 30.0 | 12.3 |
| Paclitaxel | 68.8 | 28.8 | 2.4 | 67.1 | 26.7 | 6.3 |
| Mixture of | 56.2 | 42.6 | 1.2 | | | |
| paclitaxel and | | | | | | |
| PLGA (1:1) | | | | | | |
| Mixture of | 59.1 | 40.4 | 0.60 | | | |
| paclitaxel and | | | | | | |
| PLGA (1:9) | | | | | | |
| PLGA | | | | 53.1 | 30.4 | 16.6 |
| nanospheres | | | | | | |
| with TPGS added | | | | | | |
| in water | | | | | | |
| PLGA | | | | 53.7 | 30.3 | 16.0 |
| nanospheres | | | | | | |
| with TPGS added | | | | | | |
| in oil | | | | | | |
| PLGA | | | | 45.7 | 39.4 | 14.9 |
| nanospheres | | | | | | |
| using PVA as | | | | | | |
| emulsifier | | | | | | |
| PLGA | | | | 57.5 | 28.0 | 14.5 |
| nanospheres | | | | | | |
| using TPGS as | | | | | | |
| emulsifier | | | | | | |
| Without washing | | | | | | |

(e) Surface Chemistry

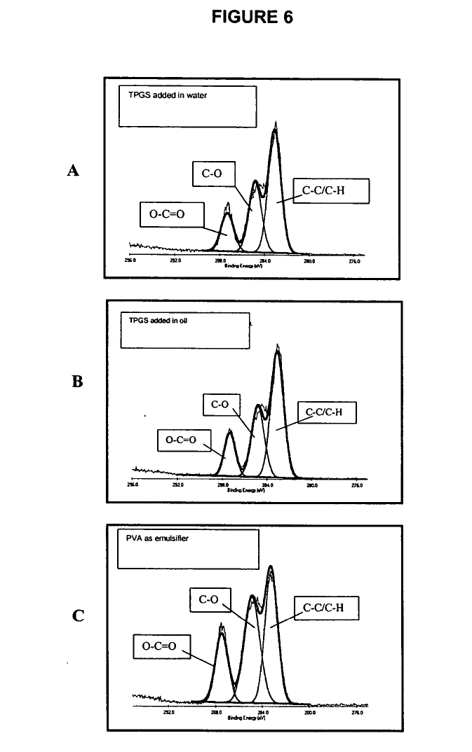

The X-ray photoelectron spectroscopy (XPS, AXIS His—165 Ultra, Kratos Analytical, Shimadzu) was utilised for analysing the surface chemistry of the nanoparticles. The angle of X-ray used in XPS analysis was 90° C. The analyser was used in fixed transmission mode with pass energy of 80 eV for the survey spectrum covering a binding energy range from 0 to 1200 eV. Peak curve fitting of the C1s (atomic orbital 1s of carbon) envelope was performed using the software provided by the instrument manufacturer. XPS is a quantitative technique that gives the elemental and averaged chemical composition by measuring the binding energy of electron associated with atoms over a 5-10 nm depth inside the polymeric matrix. The examination of XPS C1s (atomic orbital 1s of carbon) envelopes on the surface of different type of paclitaxel loaded nanoparticles was performed and the results were displayed in FIG. 6 and Table 2.

Firstly, there was no nitrogen element signal detected, which could mean that the paclitaxel was almost all distributed inside the polymeric nanoparticles (Wilcox, 1993), although the drug loading ratio was as high as 10%. To be certain, the XPS measurement of the pure paclitaxel as well as the powder mixture of the drug and the polymer was conducted. The signal of nitrogen could be detected from the mixture of various mixing ratios although it was quite low when the mixing ratio was 1:9. However, the nitrogen signal could not be detected for all types of nanoparticles. Thus, it might be concluded that the distribution of paclitaxel on the nanoparticles surface was quite rare. The best envelope fit was obtained using three main peaks corresponding to C—C/C—H (283-285 eV), C—O (285-287 eV) and O—C═O (287-289 eV) environments respectively.

By comparing the quantification summarised in Table 2, the contribution to XPS C1s envelope of carbon from the TPGS emulsified nanoparticles was similar with that of pure PLGA. However, the envelope ratio varied remarkably from the PVA emulsified nanoparticles, which differs by about 10% for both species of carbon (C—C/C—H, C—O). The investigation demonstrated that the surface chemistry of nanoparticles prepared with TPGS as surfactant stabiliser was different from that made with PVA as the stabiliser. The extra emulsifier of TPGS emulsified nanoparticles could be cleaned relatively thoroughly and there were little residual surfactants on the surface, which exceeded the detection limit of XPS analysis. Further, the XPS results were compared between the TPGS emulsified nanoparticles washed 4 times and those not washed at all during the harvesting procedure.

The amount of TPGS left on the surface of the unwashed nanoparticles could be detected significantly by XPS. The data displayed in Table 2 XPS C1s envelope from the unwashed TPGS emulsified nanoparticles were quite alike to those of pure TPGS. However, the amount of PVA left on the surface of the PVA emulsified nanoparticles could be observed. It can thus be concluded that PVA was difficult to be completely washed away from the nanoparticle surface. The remained PVA on the nanoparticle surface may have unexpected influence on the property and application of the nanoparticles and may cause side effects for human health. This is the third incomparable advantage of TPGS against the PVA as surfactant stabiliser in fabrication of polymeric nanoparticles for drug delivery.

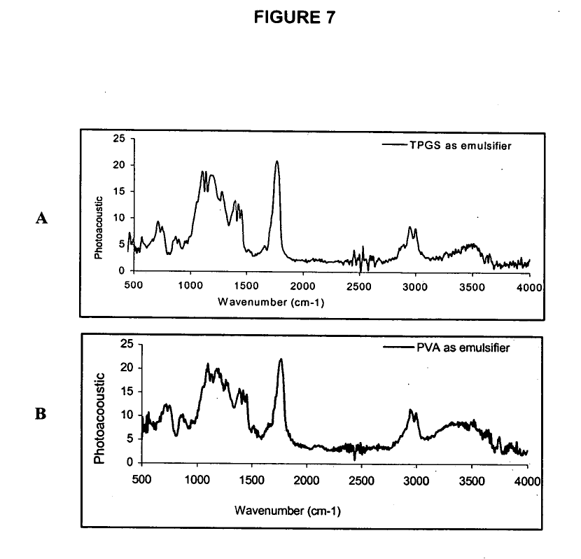

The Fourier transform infra-red (FTIR, Bio-Rad FTS-3500 FTIR, Excalibur Series, Bio-Rad Laboratories, Inc.) analysis was also conducted for the surface structure characterisation of the prepared nanoparticles with a photoacoustic spectroscopy technique (MTEC Model 300 Photoacoustic Detector System, MTEC Photoacoustic, Inc.). Nanoparticle samples were scanned in the IR range from 400 to 4000 cm −1, with a resolution of 8 cm −1 and carbon black reference. The detector was purged carefully by clean dry helium gas to increase the signal level and reduce moisture. FTIR analysis measures the selective absorption of light by the vibrational modes of specific chemical bonds in the sample. Currently, the diffusion reflectance infrared Fourier transform spectroscopy (DRIFTS) is widely being used for analysing irregular polymeric surface and the presence of specific functional groups on the graft surface. Together with the photoacoustic spectroscopy, the FTIR-PAS technique can measure a sample's absorbance spectrum rapidly and directly with a controllable sampling depth and with little or no preparation of the sample, which can be all types of solids, liquids and gases. It is operable in photoacoustic absorbance, diffusion reflectance and transmission modes, and applicable to macrosmples and microsamples. In the present work, all four recipes of prepared nanoparticles were operated by the FTIR-PAS and the obtained spectra were illustrated in FIG. 7. It showed that no significant differences of the shape and position of the absorption peaks could be observed obviously among the batches of sample. All the samples showed the main peaks contributed by the functional groups of PLGA molecule such as —CH, —CH2, —CH3 stretching (2850-3000 cm−1), carbonyl —C═O stretching vibrations (1700-1800 cm−1), C—O stretching (1050-1250 cm−1), and —OH stretching vibrations (3200-3500 cm−1) which were broad.

The spectral analysis indicated the specific functional groups of polymeric material on the surface of nanoparticles are of almost the same chemical characteristics. Although most of the absorption peaks from the PVA or TPGS emulsified nanoparticles overlapped to large extent, the characteristic peak of —CH at frequency 2850-2950 cm⁻¹ was not alike distinctly. This phenomenon may result from either the existence of trace TPGS and/or PVA on the surface of nanoparticles, which meant that there was residual surfactant left on the nanoparticles surface after the harvesting procedure, or the little influence of emulsifier used on the nanoparticles formation.

(f) In Vitro Drug Release Study

The release of paclitaxel from the nanoparticles was measured in triplicate in PBS (pH 7.4). 10 mg of paclitaxel loaded nanoparticles were suspended in 10 ml of buffer solution in a screw capped tubes and placed in an orbital shaker water bath (GFL-1086, Lee Hung Technical Company, Bukit Batok Industrial Park A, Singapore), which was maintained at 37° C. and shaken horizontally at 120 min−1. At particular time interval, the tubes were taken out of the water bath and centrifuged at 8000 rpm for 10 minutes. The precipitated nanoparticles were resuspended in 10 ml of fresh buffer before being put back in the shaker bath. The supernatant was taken for analysis of paclitaxel concentration, which was first extracted with 1 ml of DCM, followed by adding 3 ml of the mixture of acetonitrile and water (50:50, v/v), then evaporated until a clear solution was obtained under a stream of nitrogen. HPLC analysis was then conducted as previously described.

Similar to the measurement of encapsulation efficiency, the extraction procedure needs to be analyzed for the extraction recovery efficiency due to inefficient extraction. Similarly, known mass at a certain range of pure paclitaxel was dealt with the same procedure mentioned above. The determined factor was 77.5%, which meant that the obtained extraction solution only contained 77.5% of the original paclitaxel after all the related process. The data obtained for analysis of the in vitro release were corrected accordingly.

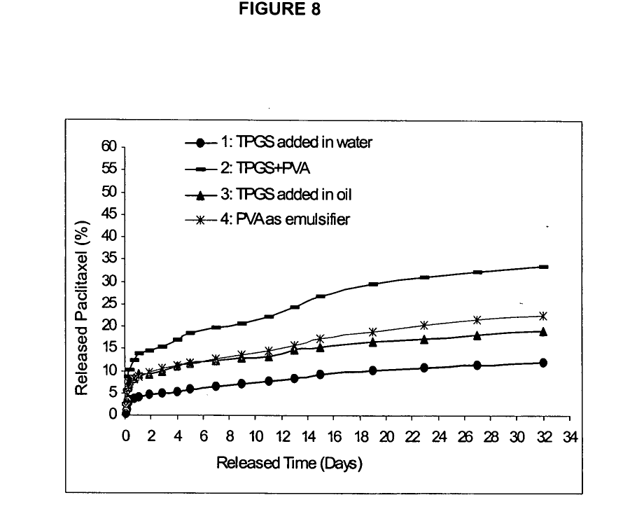

FIG. 8 showed the in vitro release curves of the four types of paclitaxel loaded nanoparticles. For all recipes, the initial burst was observed in the first day. After that the release of paclitaxel was at a constant rate. Obviously, the paclitaxel released most slowly from the nanoparticles formulated with TPGS added in the water phase in the process. When the TPGS was added in the oil phase during fabrication, the release rate became faster and nearly at the same rate as that for the PVA emulsified nanoparticles. It is interesting to notice that, when the TPGS and PVA were used together, the nanoparticles released the paclitaxel fastest. The accumulative amount of paclitaxel released after one month was about 11% for the nanoparticles fabricated with TPGS added in the water phase in the process. It was about 20% for the nanoparticles when the TPGS was added into the oil phase in the process. The accumulative amount of paclitaxel released after one month was about 22% for the nanoparticles with PVA as stabilizer and it was about 35% for the nanoparticles prepared with TPGS and PVA together as the emulsifier.

The diffusion of the drug, the erosion and swelling of polymer matrix and the degradation of polymer are the main mechanisms for the drug release. Since the degradation of PLGA is slow, the release of paclitaxel from the nanoparticles would mainly depend on the drug diffusion and the matrix erosion. In such case, the size, hardness and porosity of the nanoparticles should have significant effects on the release property. The AFM and SEM examination indicated that all types of nanoparticles had smooth surface, which supported the slow release of drug by diffusion and matrix erosion mechanism. Moreover, the size is also an important factor to determine the release rate, the nanoparticles emulsified by TPGS and PVA together were the smallest in size. Therefore the release of drug from this sample was fastest.

The other three kinds of nanoparticles had similar mean size and size distribution. They thus showed similar release rates. The reason of TPGS emulsified nanoparticles displayed slow release may come from the enhanced interaction or affinity between paclitaxel and polymer matrix. Not only does TPGS possess amphiphilic property, which is necessary for surface-active agents, but it can be dissolved in both of the oil and the water phase as well. No matter it was added in the water phase or in the oil phase, the TPGS can always be well distributed. In addition, the TPGS molecule is bulky and has large surface area. When forming the emulsion system, TPGS could have the drug and the polymer in a better contact and they can thus be blended thoroughly inside the oil phase of every droplet. Instead, PVA does not posses such a property and can thus not be distributed in the oil phase. Moreover, when TPGS was added in the water phase, the amount of the residual emulsifiers on the nanoparticle surface was found less than that on the surface of nanoparticles fabricated with TPGS added in the oil phase. Thus, the nanoparticles emulsified by TPGS added in water phase displayed slower in vitro release.

(g) Cell Uptake and Cytotoxicity of the Drug Loaded Nanoparticles

Cardiovascular smooth muscle cells (VSMC) were maintained by serial passaging in McCoy's 5A Medium supplemented with 10% fetal bovine serum (FBS), 2.2 g/L of sodium bicarbonate and 1% penicillin-streptomycin solution. Cells were cultured as a monolayer at 37° C. in a humidified atmosphere containing 5% CO2 and medium was replenished every other day. Upon reaching confluency, cells were washed twice with warm phosphate-buffered saline (PBS, pH 7.4) and harvested with 0.125% Trypsin-EDTA solution. Cells were plated at a density of 1.34×104 cells/well in 96-well plates (Costar, Corning, N.Y.) for experiments. In this study, HT-29 cells were used passages between 19 and 22. Cells were seeded at 1.34×104 cells/well in the chambered cover glass system (LAB-TEKÒ, Nalge Nunc, IL) for qualitative study or 96-well black plates (Costar, Corning, N.Y.) for quantitative analysis.

After equilibrating with Hank's Balanced Salt Solution (HBSS, pH 7.4) for 1 hr, cells were incubated with coumarin-6 loaded nanoparticle suspensions (100 μg/ml to 250 μg/ml in HBSS, pH 7.4) for 0.5, 1, 2 and 4 hrs. At the end of the experiment, cell monolayer was rinsed four times with cold PBS to eliminate the excess nanoparticles which were not taken up by the cells, and lysed with 0.5% Triton X-100 in 0.2 N NaOH. Cell associated fluorescent particles were quantified by ananlysing the cell lysate using a microplate reader (GENios, Tecan, Austria, lex 430 nm and lem 485 nm).

For the qualitative study, cells were washed four times with PBS at the end of experiment and fixed by ethanol for 20 min followed by counterstaining the nucleus with propidium iodide (PI). Then, cell monolayer was washed 2 times with PBS and mounted in Dako fluorescent mounting medium (Dako, CA) until observation by confocal laser scanning microscope (CLSM) (Zeiss LSM 410) equipped with an imaging software (Fluoview FV300).

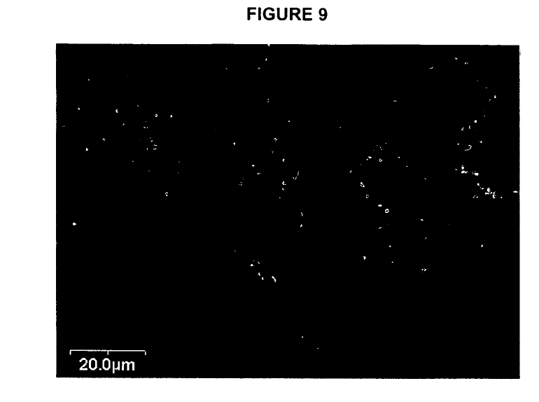

The uptake of paclitaxel loaded nanoparticles by HT-29 cells was found dependent on the size and coating material of the nanoparticles. The efficiency of uptake of TPGS-coated nanoparticles was about 6 folds higher than that of the nanoparticles without coating. Confocal microscopic studies further proved such a coating effect as shown in FIG. 9, in which the green fluorescence shows the TPGS emulsified nanoparticles taken up by VSMCs. It was further found that the density of the fluorescence is inversely proportional to the particles size, which means that the smaller the particle size, the better the cell uptake of the nanoparticles could be resulted. FIG. 9 also demonstrates that formulation of paclitaxel by vitamin E TPGS emulsified PLGA nanoparticles could be feasible for oral chemotherapy, which should be further confirmed by animal models.

To test the cytotoxicity of the drug loaded nanoparticles, VSMCs were pre-incubated with HBSS prior to experiment. Then, cells were incubated with different concentrations of paclitaxel-loaded particles or Taxol® (0.25-25 mg/ml of paclitaxel, after appropriate dilution of these formulations in 100 ml of HBSS) for 24 hours. In order to determine the cytotoxic effect of the polymer used to prepare the nanoparticles, cells were also incubated with different dilutions of the placebo nanoparticles for the same period of time. The effect of different dosage forms of paclitaxel on the cell viability was assessed by the colorimetric MTT assay. This assay is based on the cellular reductive capacity of living cells to metabolize the yellow tetrazolium salt, 3-(4,5-dimethylthizaol-2-yl)-3,5-diphenyl tetrazolium bromide (MTT), to a chromophore, formazan product, whose absorbance can be determined by spectrophotometric measurement. At the end of the experiment, cells were washed twice with PBS (pH 7.4) and further incubated with 100 ml culture medium containing 10 ml of MTT solution (5 mg/ml) for 4 h at 37° C. Isopropanol acidic solution (isopropanol-HCl 0.04 N) were then added in order to dissolve the formazan crystals formed. The UV absorbance of the solubilized formazan crystals was measured spectrophotometrically (GENios, Tecan, Austria) at 560 nm. Cell viability was determined by the ratio of Abstest cells and Abscontrol cells which represent the amount of formazan determined for cells treated with the different formulations and for control cells (non-treated), respectively.

Cytotoxic activity of paclitaxel formulated either in Cremophor EL (i.e. Taxol®) or in polymeric nanoparticles was evaluated by assessing VSMC viability by the MTT assay. A marked reduction in HT-29 cell viability was observed when the cells were exposed to TPGS coated nanoparticles, which contain paclitaxel at the same concentration with that for the other two experiments of HT-29 cells incubated with PVA emulsified nanoparticles and Taxol®. It was found that the viability of HT-29 cells after 24 hour incubation with TPGS emulsified nanoparticles is 3 times lower than that observed from the cells incubated with Taxol® in the same period. Considering that the accumulative release of paclitaxel from the TPGS nanoparticles increased from 0% to 5-8% in these 24 hours, the HT-29 cell viability caused by the TPGS emulsified nanoparticles should be 3/[(0.05˜0.08)×0.5]=75-120 times lower than that observed from the similar case administrated by Taxol®. These results demonstrated the feasibility of coating cardiovascular stents by the drug loaded nanoparticles of the present invention to achieve much higher cellular uptake of the drug and much higher VSMC mortality than the drug-eluting stents could do. Side effects can also be greatly reduced since no toxic adjuvant would be needed.

The cellular internalization of nanoparticles was confirmed by Cryo-scanning electron microscopy (Cryo-SEM). VSMCs of passage 30 were incubated with nanoparticle suspension (250 μg/ml in HBSS, pH 7.4) for 1 hour and then the excess nanoparticles were washed away with pre-warmed PBS (pH 7.4) for 3 times. Cells were fixed by using 2.5% glutaraldehyde solution and were plunged frozen in nitrogen sludge (−194° C.). The specimen was transferred to the cryo-preparation chamber of a cryo-system attached to a Philips XL30 scanning electron microscope. The temperature was raised to −95° C. The specimen was then fractured and etched for 15 min. The frozen specimen was sputter-coated with approximately 5 nm of platinum, introduced onto the specimen stage of the SEM at −130° C. and examined at 5-10 kV accelerating voltage. Cryo-SEM enables the observation of bulk biological materials in hydrated conditions by conversion of liquid water to solid by cryo-fixation, which has been widely used for ultrastructural study of biological materials and water distribution within tissues as well as for observation of ice crystal distributions following the freezing of biological materials, especially plant tissues.

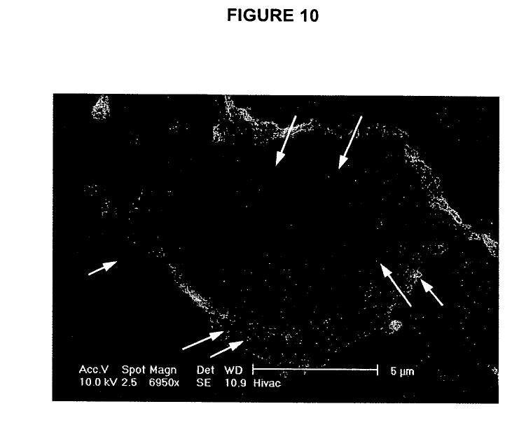

FIG. 10 shows the cryo-SEM image of a cross-section of a single Caco-2 cell after treated with vitamin E TPGS-coated PLGA nanoparticles for 1 hr at 37° C., which indeed confirms the efficient uptake and internalization of nanoparticles. The arrows indicate some of the nanoparticles found throughout the endoplasm of the cell and around the nucleus. Some nanoparticles can be found adsorbed on the cell membrane. Some free nanoparticles scattered near the cell can also be observed.

Coating of Stent with Coating of the Present Invention

(h) Formation of Lipid-Nanoparticle Monolayer at the Air-Water Interface

The Langmuir trough used for lipid monolayer formation and stent coating by nanoparticles is a Nima Langmuir-Blodgett trough, Model 601 manufactured by NIMA Technology Ltd. (The Science Park, Coventry, England). The essential features are the 105 cm² surface area trough with two mechanically coupled barriers, surface pressure sensor, sapphire window, dipper mechanism (25-mm stroke), computer interface unit IU4 and operating software (version 4.80). Lipid monolayers were spread using chloroform as solvent. Lipid stock solutions were prepared at 0.2 mM concentration. The trough was wiped using chloroform soaked Kimwipes and rinsed with Millipore water 3 times before each run. 60 ml PBS buffer was then carefully poured into the trough, ensuring that no air bubbles were formed in the process. Surface purity was checked by closing and opening the barriers and ensuring that p readings did not differ more than ±0.1 N/m.

A Hamilton syringe was cleaned 3 times with chloroform before pumping up an appropriate amount of solution into the syringe, removing all air bubbles. With the syringe just above the surface of the water, aliquots of the lipid solution were deposited drop by drop onto the aqueous surface, ensuring that surface pressure (p) returned to zero before introducing the next drop. After waiting for 25 min for solvent evaporation, compression was started at a speed of 6-7 cm2/min and the p-a isotherm was recorded. The lipid monolayer was then compressed to a desired value of surface pressure for nanoparticle penetration and stent coating.

The dipping starts either from the air phase for stents which have hydrophobic surface (e.g. polymeric), or from the water phase for those which have hydrophilic surface (e.g. metal). The dipping can be repeatedly carried out until a desired amount of drug has been contained in the multi-layers of the lipid-nanoparticle mixture. However, the dipping process should be finished with the lipid head group surface if the coated stents are to be restored in a liquid phase, or with the lipid chain layer if the coated stents are to be restored in a dry condition. It can be shown that the coated layers can be quite stable under room temperature.

(i) Nanoparticle Penetration into the Lipid Monolayer

The drug-loaded nanoparticle suspensions in PBS were prepared a concentration of 1.25 mg/ml. The nanoparticle suspension was then slowly injected into the subphase of the lipid monolayer in the Langmuir trough with a Hamilton microsyringe. Nanoparticle penetration into the lipid monolayer would spontaneously occur with surface pressure being continuously increased, which was recorded until the lipid monolayer became saturated of the nanoparticles with no further increment of the surface pressure. The total surface pressure increment, which is the substraction of the initial surface pressure from the final (saturated) surface pressure, can be related to the amount of the drug-loaded nanoparticles which have been penetrated in the lipid monolayer. It can thus be used to calculate by computer simulation the number of the drug-loaded nanoparticles within the lipid monolayer.

### REFERENCES

Dorr, R T (1994), Ann. Pharmacother, 28, S11-S14.
Huudenschild C C (1993) Am. J. Med., 94, 40S-44S.
Liistro F, Stankovic G, Di Mario C, et al. (2002) CIRCULATION, 105(16):1883-1886.
Lopes N M, Adams E G, Pifts T W, Bhuyan B K (1993) Cancer Chemother Pharmacol, 32, 235-242
Popma J J, Califf R M and Topol E J (1991) Circulation, 84, 1426-1436.
Wani M C, Taylor H L, Wall M E, Coeggon P and McPhail A T (1971) J Am Chem Soc, 93, 2325-2327.
Wilcox J N (1993) Am J Cardiol, 72, 88E-95E.
Win and Feng (2005), Biomaterials 26, 2713-2722

What we claim is:

1. A coating comprising at least one type of nanoparticle, wherein the at least one type of nanoparticle is emulsified by vitamin E TPGS and loaded with at least one drug.

2. The coating according to claim 1, wherein the at least one type of nanoparticle is biodegradeable and/or bioresorbable.

3. The coating according to claim 1, wherein the at least one type of nanoparticle is made of PLGA.

4. The coating according to claim 1, wherein the at least one drug is paclitaxel.

5. The coating according to claim 1, wherein the coating is applied to an implant.

6. The coating according to claim 5, wherein the implant is a stent.

7. The coating according to claim 6, wherein the implant is a cardiovascular stent.

8. An implant coated with at least one coating, the coating comprising at least one type of nanoparticle, wherein the at least one type of nanoparticle is emulsified by vitamin E TPGS and loaded with at least one drug.

9. The implant according to claim 8, wherein the at least one type of nanoparticle is biodegradeable and/or bioresorbable.

10. The implant according to claim 8, wherein the at least one type of nanoparticle is made of PLGA.

11. The implant according to claim 9, wherein the implant is a stent.

12. The implant according to claim 11, wherein the implant is a cardiovascular stent.

13. The implant according to claim 8, which is an implant for brachytherapy.

14. The implant according to claim 8, wherein the drug is a radioactive material.

15. The implant according to claim 8, wherein the drug is a chemotherapy drug.

16. The implant according to claim 8, wherein the drug is paclitaxel.

17. A process of coating an implant, the process comprising:
(a) providing an implant;
(b) providing a lipid monolayer comprising at least one type of nanoparticle, the nanoparticle being emulsified by vitamin E TPGS and loaded with at least one drug; and
(c) coating the implant with the lipid monolayer.

18. The process according to claim 17, wherein the at least one type of nanoparticle is biodegradeable and/or bioresorbable.

19. The process according to claim 17, wherein the at least one type of nanoparticle is made of PLGA.

20. The process according to claim 17, wherein the at least one drug is paclitaxel.

21. The process according to claim 17, wherein the implant is a stent.

22. The process according to claim 21, wherein the implant is a cardiovascular stent.

23. A method of controlling and/or reducing minimize restenosis and/or multi-drug resistence in a subject receiving a cardiovascular stent, the method comprising implanting a cardiovascular stent coated with at least one coating comprising at least one type of nanoparticle made of a biodegradeable and/or bioresorbable polymer, the at least one type of nanoparticle being emulsified by vitamin E TPGS and loaded with paclitaxel.
